(19) United States
(12) Patent Application Publication
(10) Pub. No.: US 2002/0128258 A1
(43) Pub. Date: Sep. 12, 2002

(54) Therapeutical method involving subcutaneous administration of drugs containing cephalotaxine derivatives

(76) Inventors: Jean-Pierre Robin, Trange (FR); Jean-Pierre Marie, Sevres (FR); Nina Radosevic, Trange (FR); Julie Blanchard, Rouillon (FR)

Correspondence Address: Norman H. Stepno, Esquire, BURNS, DOANE, SWECKER & MATHIS, L.L.P., P.O. Box 1404, Alexandria, VA, 22313-1404, US

(21) Appl. No.: 09/801,751

(22) Filed: Mar. 9, 2001

(51) Int. Cl. A61K 31/55

(52) U.S. Cl. 514/214.01

(57) ABSTRACT

A new method of therapy using the subcutaneous mode of administration of formulations based upon harringtonines including their salts and tautomeric forms having the formula

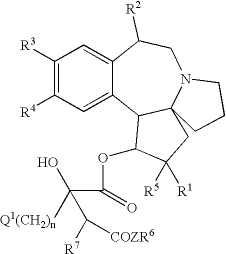

where:

R¹ is H, OH, OMe, O—(C₁-C₃₀)alkyl, O-aryl-(C₁-C₃₀)-alkyl, O—(C₂-C₃₀)-alkenyl, O—(C₃-C₃₀-cycloalkyl or null and

R² is H or OH, or R¹, R² form together —O—.

R³ ═R⁴ 50 OMe or R³ and R⁴ form together —OCH₂O—,

n is 0 to 8,

R⁵ is H, OH, OMe, O—(C₁-C₃₀)-alkyl, O-aryl-(C₁-C₃₀)-alkyl, O—(C₂-C₃₀)-alkenyl, O—(C₃-C₃₀)-cycloalkyl or O-aryl,

Z═O, S, or NH, and

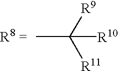

or Z—R⁸ is NR¹²R¹³, R¹² and R¹³ representing respectively R⁹ and R¹⁰,

R⁹, R¹⁰, R¹¹ are independently H, C₁-C₃₀ alkyl, C₃C₃₀ cycloalkyl, aryl, aryl-(C₁-C₃₀)-alkyl, C₂-C₃₀ alkenyl, C₂-C₃₀ alkynyl, C₁-C₃₀trihalogenoalkyl, C₁-C₃₀alkylamino-(C₁-C₃₀)alkyl, C₁-C₃₀ dialkylamino(C₁-C₃₀)-alkyl, or amino-(C₁-C₃)-alkyl, or

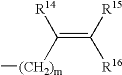

where R¹⁴, R¹⁵, R¹⁶ are independently H, halogen, C₁-C₃₀ alkyl, C₃-C₃₀ cycloalkyl, aryl, aryl-(C₁-C₃₀)-alkyl, C₂-C₃₀ alkenyl or C₂-C₃₀ alkynyl, C₁-C₃₀trihalogenoalkyl, m is 0 to 4, each of these groups including or not heteroatom(s)

or their combination with another antitumor agent or a mixture of antitumor agents useful for the treatment of a disease in humans or animals, particularly cancers, leukemias, lymphomas, parasite diseases or chemotherapeutic resistance to other agents, In using a formulation specifically adapted for subcutaneous administration.

### BACKGROUND

Harringtonines (i.e. harringtonine=HA and homoharringtonine=HHT) are particular cephalotaxine esters, alkaloids isolated from rare and endangerous conifers belonging to the Cephalotaxus genus. Cephalotaxine and its natural ester are gathered under the generic term of cephalotaxane.

### Definitions ( See Following Scheme 1)

Cephalotaxanes are particular alkaloids today only extracted from the Cephalotaxaceae family which exhibiting the structural formula 1. Several substituants may be encountered on this core structure; hydroxyl, ether, acyloxy etc. The eventual presence of some additional double bound or intramolecular bridge achieve to definite cephalotaxanes. Cephalotaxines 2 and harringtonines 3, are examples of cephalotaxanes. Several dozen of cephalotaxanes have been isolated from various Cephalotaxus species.

Cephalotaxanes are unnatural structural analogs of cephalotaxanes.

Cephalotaxoids is a generic term which groups together cephalotaxane and neocephalotaxanes

Cephalotaxines 2 are cephalotaxanes without acyloxy side-chain. Cephalotaxine 2a and drupacine 2b are example of cephalotaxines.

Harringtonines 3 are particular cephalotaxanes formed by attachement of a branched hydroxyacyloxy side-chain at the 3-position of various cephalotaxines moieties. Harringtonines are natural esters of cephalotaxines exhibiting generally a strong cytotoxic activity. However the lost only one atom of this minimal structure lead to a dramatic lost of activity (see below). Some example of harringtonines are harringtonine 3a, homoharringtonine 3b, drupangtonine 3c, anhydroharringtonine 3d and neoharringtonine 3e.

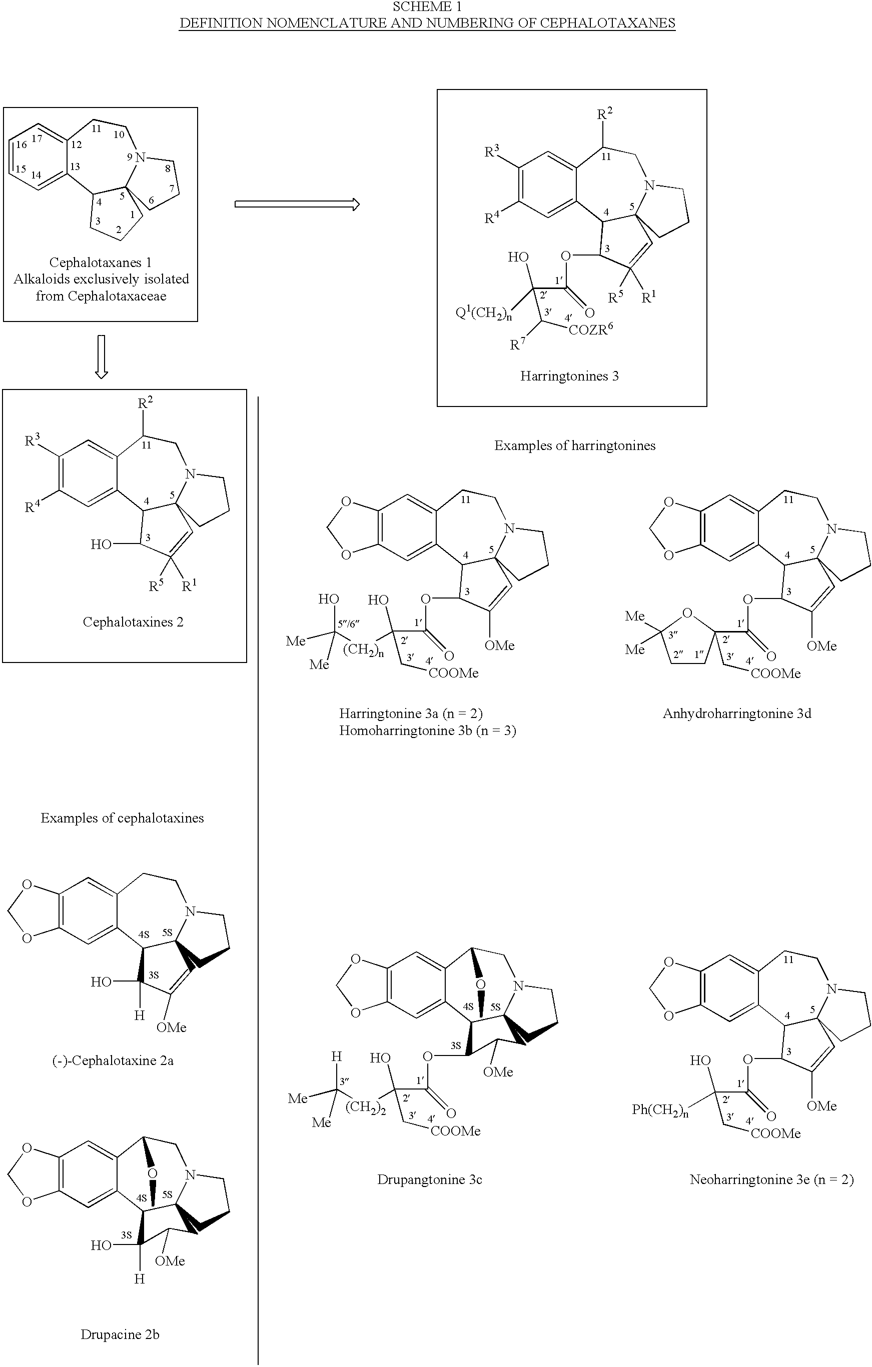

in the above formulae 3 and 3 the different substituants have the following definitions:

R¹ is H, OH, OMe, O—(C₁-C₃₀)-alkyl, O-aryl-(C₁-C₃₀)-alkyl, O—(C₂-C₃₀)-alkenyl, O—(C₃-C₃₀)-cycloalkyl or null and

R² is H or OH, or R¹, R²form together —O—.

R³═R⁴═OMe or R³ and R⁴ form together —OCH₂O—,

n is 0 to 8,

R⁵ is H, OH, OMe, O—(C₁-C₃₀)-alkyl, O-aryl-(C₁-C₃₀)-alkyl, O—(C₂-C₃₀)-alkenyl, O—(C₃-C₃₀)-cycloalkyl or O-aryl,

Z═O, S, or NH, and

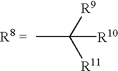

or Z—R⁸ is NR¹²R¹³, R¹² and R¹³ representing respectively R⁹ and R¹⁰,

R⁹, R¹⁰, R¹¹are independently H, C₁-C₃₀ alkyl, C₃-C₃₀ cycloalkyl, aryl, aryl-(C₁-C₃₀)-alkyl, C₂-C₃₀ alkenyl, C₂-C₃₀ alkynyl, C₁-C₃₀trihalogenoalkyl, C₁-C₃₀alkylamino-(C₁-C₃₀)alkyl, C₁-C₃₀ dialkylamino(C₁-C₃₀)-alkyl, or amino-(C₁-C₃₀)-alkyl, or

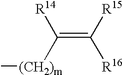

where R¹⁴, R¹⁵, R₁₆ are independently H, halogen, C₁-C₃₀ alkyl, C₃-C₃₀ cycloalkyl, aryl, aryl-(C₁-C₃₀)-alkyl, C₂-C₃₀ alkenyl or C₂-C₃₀ alkynyl, C₁-C₃trihalogenoalkyl, m is 0 to 4,

each of these groups including or not heteroatom(s).

### Introduction

Cephalotaxine esters such as harringtonines [i.e. homoharringtonine (HHT) and harringtonine (HA)] were first investigated in China and reported to be active in myeloid leukemias, including Acute NonLymphocytic Leukemias (ANLL) or Acute Myeloid Leukemias (AML)[1]. Early Phase I and II studies confirmed its antileukemic activity but documented a high incidence of cardiovascular toxicity with short-infusion schedules,[2, 3] and with higher-dose continuous infusion schedules.[4] However, definite activity was observed in AML, acute promyelocytic leukemia (APL), and myelodysplastic syndrome (MDS) including AML related MDS.[4-7] A lower-dose longer-duration continuous infusion schedule of HHT (2.5 mg/m² daily for 10 to 14 days instead of 5 to 9 mg² daily for 5 to 7 days) abrogated the occurrence of cardiovascular complications including hypotensive events and arrythmias which occurred in less than 5% of patients with the new schedule.[7] This observation, together with the noted antiproliferative effect of HHT, resulted in further studies of the new schedule in CML. Subsequently, the efficacy of HHT alone in patients in late chronic phase CML (duration of disease more than 12 months), many of whom were interferon-alpha (IFN) resistant, and in sequence with IFN In early chronic phase CML was reported. [8, 9] In both studies, significant anti-CML efficacy was observed. It should be pointed out that numerous studies using HHT alone or in multiple combinations, In the setting of myeloid leukemias, were carried out in China these past two decades.[10-38]

Cytarabine (ara-C) has shown activity in CML as a single-agent, and in combination with IFN.[39-41] HHT has also been reported to be synergistic with ara-C and/or with IFN in preclinical models .[42, 43] The limited therapeutic options of patients in late chronic phase CML and the efficacy of both HHT and ara-C in vitro[42-46] and in vivo,[8, 9, 39-41] led to a recent successful investigation of HHT and low-dose ara-C combination in patients who have failed IFN regimens and in de novo patients.[43, 47-50] Among patients treated in chronic phase, 72% achieved CHR; 31% achieved a cytogenetic response, which was major in 14% of the cases. Considering that the study group included mostly IFN resistant patients in late chronic phase who had few therapeutic options available, the median duration of disease control of 10 months, and estimated 4-year survival rate of 55% were favorable.[50] Comparable, or even better results, have also been reported by Ernst et al.[43, 47-49] using HHT 2.5 mg/m² daily and ara-C 7.5 mg/m² daily by continuous infusion for up to 14 days. In their report, the CHR rate among 44 patients treated was 93%, and cytogenetic responses were observed in 16 of 36 patients (44%) treated for at least six months. Their study group included 14 patients in early chronic phase CML: all 14 (100%) achieved CHR, and 11 of 13 evaluable (84%) had a major cytogenetic response. Moreover, triple combination therapy using low-dose of HHT plus ara-C plus IFN in the setting of de novo patients with CML, gave very encouraging responses.[51] A number of Chinese studies used HHT plus intermediate dose of ara-C, vincristine (Oncovin) and prednisone (treatment called HOAP) in AML and CML.[10-18]. Only one U.S. study used HHT combined with cytarabine given at intermediate dose.[52, 53]. In conclusion, the combination of HHT with subcutaneous cytarabine is practically always recommended by the medical community where HHT alone is efficient.

In addition, Sacchl et al. [54] recently pointed out that combinations of HHT with another nucleoside such as 5-azacytidine (decitabine) is very promising in the treatment of advanced phases of CML. STI571

As recently stated by Kantarjian et al. [50] the route-schedule delivery of harringtonines may expand their potential use in hematologic and even In solid tumors.[55] The continuous infusion (Cl) schedule, while effective against CML, is cumbersome and limits the investigation of even lower-dose longer-exposure schedules (e.g. 0.5 to 1 mg/m² for 3 to 4 weeks). A safe subcutaneous (SQ) schedule could allow reinvestigating harringtonines, not only in CML, but also as maintenance therapy in AML. as differentiation therapy in APL, and as a chronic subcutaneous low-dose schedule in MDS.

In summary, if it can be demonstrated that the SQ mode of administration is possible such discovery could rejuvenate/revive anti-cancer research involving harringtonines.

Until now, a natural version of HHT has been used by the U.S. National Cancer Institute (NCI) in its clinical trials. Surprisingly, although more than thirty studies involving HHT have been performed, the subcutaneous mode of administration was never used in any of them.. It should be pointed that before 1985 the base form of alkaloid homoharringtonine was used for animal screening and in early studies in humans in the U.S. Since 1985 an acidic preparation bearing a pH ranging from 3 to 5.5 has been used for all clinical trials performed under the auspices of NCI.

In summary, until now the use of harringtonines by weekly or more continuous intravenous infusion has lead to a number of disadvantages which have prevented its large scale use. Such drawbacks include:

General infections, mainly frequent septicemia due to direct introduction of germs by catheter systems

Need of highly trained personel and often even hospitalization of patients for the application of the therapy

Difficulties to use such drugs using very low doses permanently

Difficulties to use such drugs in the case of elderly and younger patients.

### DESCRIPTION OF THE INVENTION

The present invention describes a new method of therapy, its use/application in human and animal diseases and disorders, particularly cancers, leukemias, lymphomas, parasite diseases and therapeutic resistance to other agent, by the subcutaneous mode of administration of a drug based upon harringtonines such as homoharringtonine or harringtonine their salt and tautomeric form eventually combined with one or more chemotherapeutic agents or inhibitor of resistance, using a specifically adapted formulation in which (i) the pH of the formulation or constituted solution for injection ranges between 5.5 and 8.5, (ii) the harringtonines are solution or hydrophilic freeze-dried powder ready-to-reconstitute of buffered salt of homoharringtonine or harringtonine and, (iii) the level of chromatographic purity of harringtonines suitable for pharmaceutical use is higher than 99.7%.

An important aspect of this invention is that the formulation of salt form of harringtonines administrated in mammals by the subcutaneous mode of administration has had much better bioavailability than the base form of the alkaloids harringtonines used in early clinical trials.

Another aspect of the invention is that harringtonines may be combined in the same subcutaneous injection with another chemotherapeutic agent, such as cytosine arabinoside, azadeoxycytidine (decitabine)or troxacytabine.

Further another aspect of the invention is that the subcutaneous mode of administration can be performed by bolus injection at regular intervals such as one to four injection a day during 1 to n days for a cycle of n days, n being preferably 28, or by continuous subcutaneous infusion.

The main advantage of this invention is the excellent local tolerance of the drug administrated subcutaneously. Simultaneously, and due to the excellent bioavailability discovered in animal experiences, the durability of the therapeutic efficacy is expected, parrticularly against leukemias, compared to the existing continuous intravenous mode of administration.

Another advantage of this invention is to improve the quality of life of the patient (absence of permanent infusion system), especially when the new method of therapy is applied to outpatients, older and/or young patients.

Also, another great advantage of this invention is the self-administration method or administration of drugs based upon harringtonines by persons with minimal medical training such as the family of the patient.

An additional advantage of the new method of administration is extending the use of drugs based upon harringtonines to animal cancers and leukemias which is not easy using the standard continuous infusion method.

Another aspect of the invention is that the application of the new method of treatment reduces the risk of general infections such as septicemias.

An additional aspect of the invention is that it does not necessitate the use of additional administration operation when harringtonines are given in combination with subcutaneous cytarabine as a compatible mixture.

Yet, another advantage of the invention for the patient is its lower cost ( absence of additional costs related to the existing complex delivery systems such as electronic pump, disposable continuous infusion systems and hospitalization)

Yet another aspect of the invention is the preparation of a new family of stable formulations of harringtonines exhibiting a weak potential for skin irritation, which would permit a safer long-term use of the drug.

### EXAMPLES

### Example 1

### Preparation of HHT Hydrochloride Drug Product for Subcutaneous Injection

Highly purified (HPLC purity 99.95%) crystalline HHT drug substance (150 grams) is dissolved by mixing in acidified (one equivalent of hydrochloric acid) water for injection acidified by until total disappearance of solid phase. Pharmaceutical grade mannitol (300 g) is dissolved, then pH is adjusted to between 5.5 and 7 with 0.01 N HCI or with 1% sodium bicarbonate, then the clear resulting solution is sterilized by filtration. After analytical controls, 30,000 5-mL sterile vials are filled with the preceding sterile bulk drug solution and submitted to lyophilisation (−40° C., 0.05 millibar), After usual sealing, labelling and control the batch was released. All operations are under control according to the current Good Manufacturing Process (cGMP) for parenteral drugs required by the United States Food and Drug Administration (FDA).

### Example 2

### Comparative Pharmacokinetic Study in Dogs After Subcutaneous Injection of 5 mg/m2 of Hydrochloride and Base Forms of HHT

### SUBACUTE TOXICITY AND PHARMACOKlNETlC SCREENING OF TWO FORMS OF HHT

### Species/Strain: Beagle Dog

Number of animals/sex/dose: the same dog was used for each treatment

Dosing:

treatment 1: 0.25 mg/kg of substance A (HHT-hydrochloride form)

treatment 2: 0.25 mg/kg of substance A (HHT-hydrochloride form)

treatment 3: 0.25 mg/kg of substance B (HHT-base form)

Mode:

treatment 1: Intravenous infusion (IV)

Treatments 2 and 3: subcutaneous injection (SQ)

Schedule:

treatment 1: continuous Intravenous infusion (CIVI) for 48 hours (days 1 and 2)

treatment 2: single subcutaneous (SQ) injection on day 5

treatment 3: single subcutaneous (SQ) injection on day 11

Duration of treatment: see § administration/day

Formulation:

treatments 1 and 2: sterile isotonic saline solution

Treatment 3: aqueous solution in 0.5% hydroxyethylcellulose with 0.1% Tween 80

Analytical method: HPLC with flourimetric detection

### Clinical Examination

Treatment 2:

yellowish vomiting, soft feces, transient decrease in activity and hypersalivation,

Treatment 3:

liquid feces with presence of blood, hypersalivation, decrease in activity, pallor of gingival mucous membranes and decrease in activity. Following these signs, the decision was taken to sacrifice the dog on ethical grounds on day 11.

Body weight

After treatment 1: body weight loss of 1.1 kg,

After treatment 2: body weight loss of 0.4 kg.

Pathology

macroscopic post-mortem examination: thickened, edematous and reddish colored thymus, firm consistency of heart, many greyish foci on liver, several greyish nodules on spleen together with irregular surface, enlargement of kidneys together with several greyish foci, dilatation of ureters together with reddish contents, thickened mucosa of urinary bladder together with many reddish foci, reddish colored mucosa of the stomach and intestines together with thick reddish contents,

microscopic examination of the skin biopsy (taken on day 6); the test substance was well tolerated at the local level.

Toxicology Conclusion

The test substance HHT, when administered to one dog by continuous intravenous infusion for 48 hours (as HHT-hydrochloride) or by subcutaneous injection (as HHT-hydrochloride) and HHT-base) at the dose-level of 0.25 mg/kg, resulted in good tolerance. Second subcutaneous injection (HHT-base form) result in mortality 8 hours after the beginning of the treatment. It was not possible to conclude if mortality was caused by cumulative toxicity or specific toxicity of the base form of HHT. Prior to death, the principal clinical signs included liquid feces, hypersalivation, decrease in activity and pallor of gingival mucous membrane.

No clinical signs were noted following the CIVI. However, the dog showed a body weight loss following the IV and first SQ treatments with the test substance A (HHT-hydrochloride).

Histopathology of a skin biopsy taken the day after the SQ injection of HHT-hydrochloride demonstrated that the test substance was well tolerated at the local level.

### Pharmacokinetic

Blood samples were collected at time 0, 30, 60, 120, 240, 480 minutes and 24 hours then HHT concentration was determined in plasma by an HPLC method in using a fluorimetric detection. The evolution of concentration In function of time was plotted as showed in below figure. Maximum of concentration reached 112 ng/mL after 2 hours for HHT hydrochloride versus only 18 ng/mL after 0.5 hours for the base form of the alkaloid. Area under the curve (AUC) was 280 ng/mL×hr for HHT hydrochloride versus only 86.5 ng/mL×hr for HHT-base, Calculated rate AUC_{HHT-hydrochloride}/AUC_{HHT-base} was 3.24.

Pharmacokinetic Conclusion

After subcutaneous injection in dogs, the bioavailability of salt form of HHT is considerably higher (triple) than the base form of this alkaloid. This finding combined with time and level of blood concentration indicates that subcutaneous mode of administration of HHT hydrochloride compatible with human use

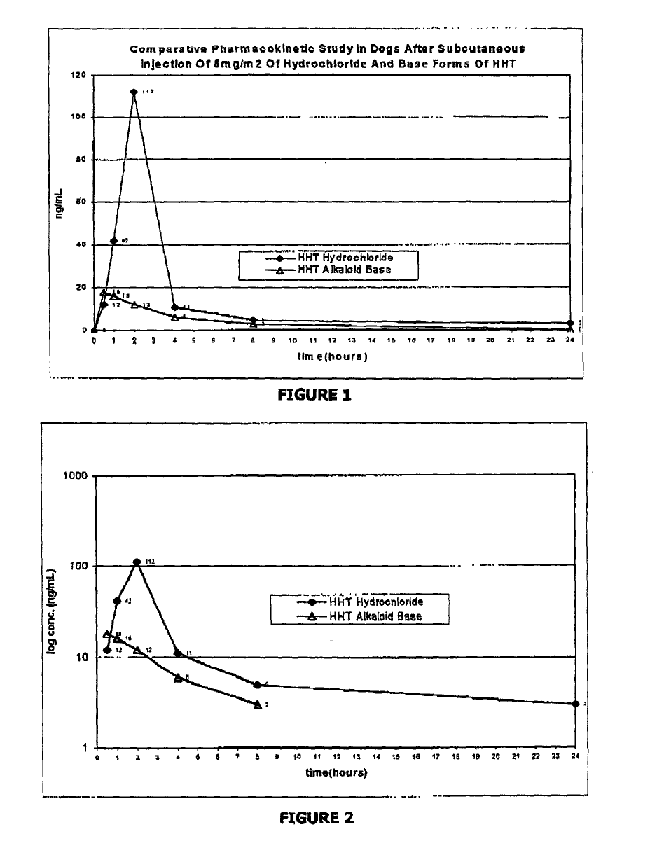

The results of the pharmacokinetic study are illustrated in the appended FIGS. 1 and 2.

### Example 3

### Investigational Treatment a Patient (No OP-99-04 #01) with an Acute Leukemia by Subcutaneous Injection of Low-Dose HHT Hydrochloride

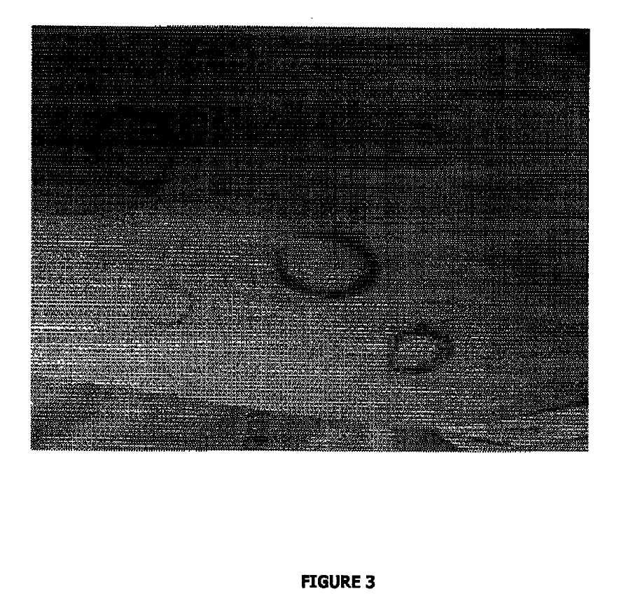

M. Mont. (No OP-99-04 #01) was a patient with an acute myeloid leukemia resistant to all available approved and investigational chemotherapies. In addition, this patient was not eligible for bone marrow transplantation. All current good clinical practices and applicable ethical rules were applied. Particularly, a written treatment protocol had been approved by an institutional review board and the patient had signed a consent form before its enrollment in the clinical trial performed in a French institution located in Paris. Physicians of this institution were experienced with the clinical use of the same formulation by intravenous mode of administration in patients with myeloid leukemias. Before starting the investigational treatment based upon the subcutaneous mode of administration, all routine clinical, biochemical and biological investigations including bone marrow aspiration were performed to confirm the diagnosis, the clinical characteristics of the patient and also to check any possible contra-indication. A 5 mg-vial of lyophilized HHT hydrochloride for injection was reconstituted with 2.5 mL of saline water for injection. A volume corresponding to 1 mg of HHT per square meter of body surface area per day divided In two daily subcutaneous injections (2 subcutaneous injections a day) was administered in a short single injection, during a period of nine (9) consecutive days. During this period no local sign of intolerance was encountered at the point of injection, Moreover, the injection was indolent and neither local signs such as inflammatory reaction, induration, tumefaction, pruritus, nor general clinical signs were experienced by the patient. For ethical reasons, and due to a total absence of clinically detectable reactions, a biopsy for microscopic analysis was not performed. The appended FIG. 3 illustrates the patient's forearm 3 days after five consecutive subcutaneous injections in five dose skin areas (small hematomas, sometimes seen at the point of injection, are caused by thrombocytopenia and are not drug related):

Periphera and white blood cells and platelet counts indicated a relatively fast decrease of granulocyts count. Post-induction treatment follow-up indicated a strong myelosuppressive effect typical of activity of intravenous HHT usually encountered in using a dose of 2.5 mg/m²/day. No additional side-effects or detectable signs of toxicity were experienced by the patient during the treatment. Accordingly, it was concluded that subcutaneous HHT Is at least as safe and efficient as intravenous HHT.

### Example 4

### Investigational Treatment a Patient (No OP-99-04 #02) with an Acute Leukemia by Subcutaneous Injection of Low-Dose HHT Hydrochloride

M. Mor. (No OP-9904 #02) was a patient with a blastic phase of chronic myelogenous leukemia resistant to all available approved and investigational chemotherapies and was not eligible for bone marrow transplantation. After using the same method of therapy than in example 3, no local sign of intolerance was encountered at the various points of injection. As for the patient of example 3 the injection was indolent and neither local signs such as inflammatory reaction, induration, tumefaction, pruritus, nor general clinical signs were experienced by this patient. For ethical reasons, and due to a total absence of clinically detectable reactions, a biopsy for microscopic analysis was not performed. More generally, same clinical features as in example 3 were found.

### Example 5

### Investigational Treatment a Patient (No OP-99-04 #03) with an Acute Leukemia by Subcutaneous Injection of Low-Dose HHT Hydrochloride

M. X. (No OP-99.04 #03) was a patient with an acute myeloid leukemia resistant to all available approved and investigational chemotherapies and was not eligible for bone marrow transplantation. After using the same method of therapy than in example 3 and 4, no local sign of intolerance was encountered at the various points of injection. As for the patient of example 3 and 4 the injection was indolent and neither local signs such as inflammatory reaction, induration, tumefaction, pruritus, nor general clinical signs were experienced by this patient. For ethical reasons, and due to a total absence of clinically detectable reactions, a biopsy for microscopic analysis was not performed. Same clinical improvements as those seen in example 3 and 4 was found.

### Bibliography

1. Anonymous and C.R.C. GROUP, Cephalotaxine esters in the treatment of acute leukemia. A preliminary clinical assessment. Chin Med J (Engo, 1976. 2(4); p. 263-72.

2. Legha, S. S., et al., Phase I clinical investigation of homoharringtonine. Cancer Treat Rep, 1984. 68(9); p. 1085-91.

3. Neidhart, J. A., et al, Phase I trial of homoharringtonine administered by prolonged continuous infusion. Cancer Res, 1986. 48(2): p. 967-9.

4. Warrell, R. P., Jr., C. J. Coonley, and T. S. Gee. Homoharringtonine: an effective new drug for remission induction in refractory nonlymphoblastic leukemia. J Clin Oncol, 1985. 3(5): p. 617-21.

5. Feldman, E., et al., Homoharringtonine is safe and effective for patients with acute myelogenous leukemia. Leukemia, 1992. 6(11): p. 1185-8.

6. Feldman, E. J., et al., Homoharringtonine in patients with myelodysplastic syndrome (MDS) and MDS evolving to acute myeloid leukemia. Leukemia, 1996. 10(1) p. 40-2.

7. Kantarjian, H. M., et al, Phase II study of low-dose continuous infusion homoharringtonine In refractory acute myelogenous leukemia. Cancer, 1989. 63(5). p. 813-7.

8. O'Brien, S., et al., Homoharringtonine therapy induces responses in patients with chronic myelogenous leukemia in late chronic phase. Blood, 1995. 86(9); p. 3322-6.

9. O'Brien, S., et al. Sequential homoharringtonine and interferon-alpha in the treatment of early chronic phase chronic myelogenous leukemia. Blood, 1999. 93(12): p. 4149-53.

10. COLLEGE., S. M.. Analysis of 51 Cases of Acute Non-lymphocytic Leukemias Treated with Combined Program of Chinese Traditional Medicine and HOAP. Chung Hua Nei Ko Tsa Chih, 1981. 20(4): p. 205-7.

11. Anonymous and S. M. College, High remission induction (traditional sino-western HOAP) regimen for acute nonlymphocytic leukemia. Chin Med J (Engl), 1980. 93(8): p. 565-8.

12. Hua, Z. and S. Xiong, Combined HOAP and traditional Chinese medicine in treating acute nonlymphocytic leukemia. Chung Hua Hsueh Yeh Hsueh Tsa Chi, 1982. 3: p. 296-298.

13. Group, S.L.R.C., Clinical study of cephalotaxine ester in the treatment of acute non-lymphoid leukemia. Shanghai Med J, 1983. 6: p. 319-323.

14. Shan, Y. D., [HOAP/HOAGP in the treatment of acute non-lymphocytic leukemia: an analysis of 68 cases]. Chung Hua I Hsueh Ts Chih, 1985. 65(10): p. 590-2.

15. Zhang, Z. Y., et al., Curative effect of harringtonine semisynthetic harringtonine and HOAP on nonlymphocytic leukemias. Analysis of 304 cases. Chin Med J (Engl), 1987. 100(7): p, 565-8.

16. Zhang, Z. Y., C. H. Hou, and Y. F. Zhu, A preliminary therapeutic analysis of 82 cases of chronic granulocytic leukemia treated with harringtonine. Chung Hua Nei Ko Tsa Chih (Chinese J Intern Med), 1986. 25(3): p. 156-7, 190.

17, Zhang, G. Z., [Combination treatment of acute non-lymphocytic leukemia with HOAP and AA-preliminary report of 14 cases]. Chung Hua Chung Liu Tsa Chih, 1991. 13(1): p. 52-4.

18. Meng, F., B. Xu, and S. Zhou, Harringtonine combined arabinoside with etoposide or VM26 for treatment refractory acute non lymphocytic leukemia. Blood, 1998. 92(10) Suppl. 1 Abstract (#3925).

19. Pan, R. P., Analysis of 33 cases of acute promyelocytic leukemia and the therapeutic effects of harringtonine treatment, Chung Hua Hsueh Yeh Hsueh Tsa Chi, 1981.2: p.24-26.

20. Anonymous and L.R.G.O.T.C.L.A.T. HOSPITAL, An analysis of 72 cases of leukemia treated with Cephalotaxus ester alkaloid. Zhonghua Yixue Zazhi [Journal of the Chinese Academy], 1978. 58: p. 163-166.

21. Bian, S. G., Y. S. Hao, and Z. C. Wang, [Analysis of the therapeutic efficacy and prognostic factors of intensive chemotherapy in 91 patients with acute nonlymphoblastic leukemia]. Chung Hua Nei Ko Tsa Chih, 1990. 29(1): p 22-5, 60.

22. Grem, J. L., et al., Cephalotaxine esters: antileukemic advance or therapeutic failure? J Natl Cancer Inst, 1988. 80(14): p. 1095-103.

23. Zhang, Z. and C. Hou, Clinical analysis of the therapeutic effects of semisyn-thetic harringtonine in treating 55 cases of non-lymphocytic leukemia. Chinese J Intern Med 1981;20;667-669. Chinese J Intern Med. 1981. 20: p. 887-669.

24. Li, L. H., et al, Clinical study of Cephalotaxus ester in the treatment of polycythemia vera. Chinese J Intern Med. 1984, 23 :p.:413-415.

25. Li, Y. H., et al,, Combined chemotherapy with harringtonine for treatment of 25 cases of acute non-lymphocytic leukami. Chung Hua Erh K O Tsa Chi, 1981. 12: p. 231-233.

26. Li, Y. H., et al., Combined harringtonine or homoharringtonine chemotherapy for acute nonlymphocytic leukemia in 25 children. Chin Med J (Engl), 1983. 96(4): p. 303-5.

27. Bian, S., et al., Comparison of four regimens composed of three drugs in treatment of untreated acute adult myeloid leukemia. Blood, Suppl. 1; abstract 1407, 2000.

28. Sun, Y., Current status of research on new anticancer drugs in China. Gan To Kagaku Ryoho, 1992. 19(8 Suppl): p. 1126-33.

29. Mi, Y., et al. EXPLORE OF POSTREMISSION THERAPY IN ACUTE MYELOID LEUKEMIA. Blood, . Abstract 4647.

30. Anonymous and C.L.A.T. HOSPITAL, Harringtonine in acute leukemias. Clinical analysis of 31 cases. Chin Med J (Engl), 1977. 3(5): p. 319-24.

31. Lu, L. H., et al., Harringtonine in treatment of polycythemia vera. Chin Med J (Engl), 1983. 96(7): p. 533-5.

32. Guo, A. X., S. L. Huang, and Q. E. Wang, [HATP chemotherapy combined with Chinese traditional medications in treating acute promyelocytic leukemia]. Chung Hua Nei Ko Tsa Chih, 1993. 32(7): p. 470-2.

33. Hou, C. H. and Z. Y, Zhang, [Intrathecal injection of harringtonine and homoharringtonine in treating central nervous system leukemia-clinical analysis of 26 cases (author's transl)]. Chung Hua I Hsueh Tsa Chih. (J Chinese Acad 1981), 1981. 61(9): p. 530-2.

34. Lu, L. S. and J. X. Chen, Leukemia Coordination Group. Homoharringtonine and harringtonine in acute nonlymphocytic leukemia: clinical observa-tions of 40 cases. Chinese J Intern Med. 1978. 17; p. 162-164.

35. Dong, Z. R., E. G. Yao, and S. R. Xu, [Low-dose ARA-C and HOAAP regimen in the treatment of adult acute nonlymphocytic leukemia; analysis of 59 cases]. Chung Hua Nei Ko Tsa Chih, 1988. 27(5): p. 300-2, 327.

36. Ye, J. S., et al., Small-dose Harringtonine induces complete remission in patients with acute promyelocytic leukemia. Leukemia, 1988. 2(7); p. 427-9.

37. Anonymous and L.R.S. CHINESE PEOPLE'S LIBERATION ARMY 187TH HOSPITAL, The therapeutic effectiveness study of 99 leukemic patients treated with four alkaloids of Cephalotaxus fortunei Hook f. China Herbal Medicine Report, 1977. 5:12: p. 25-29.

38. Zhang, Z., Treatment of acute leukemia with harringtonine, semisyn-thetic harringtonine or total alkaloids of Cephalotaxus hainanensis: analysis of curative results in 46cases. 1981;19: abstr233. Chung Hua Erh K O Tsa Chi, 1981. 19 abs. 223.

39. Sokal, J. and S. H. Beingner, Low-dose cytosine arabinoside by subcutaneous infusion in early and advanced chronic granulocytic leukemia. Blood, 1986. 68:233a (abstract).

40. Robertson, M. J., et al., Hematologic remission and cytogenetic improvement after treatment of stable-phase chronic myelogenous leukemia with continuous infusion of low-dose cytarabine. Am J Hematol. 1993. 43(2): p. 95-102.

41. Kantarjian, H. M., et al., Treatment of advanced stages of Philadelphia chromosome-positive chronic myelogenous leukemia with interferon-alpha and low-dose cytarabine. J Clin Oncol, 1992. 10(5): p.772-8.

42. O'Brien. S., et al, Homoharringtonine induces apoptosis in chronic myelogenous leukemia cells. Blood, 1993. 82(Suppl. 1): Abstract 555a.

43. Ernst, T. J. and S. Cha, Homoharringtonine and cytarabine are both selective and synergistic in inhibiting the growth of CML CFU in vitro. Blood, 1994. 84 suppl. 1 abs 151.

44. Sokal, J. E., S. S. Leong, and G. A. Gomez, Preferential inhibition by cytarabine of CFU-GM from patients with chronic granulocytic leukemia. Cancer, 1987. 59(1): p. 197-202.

45. Wachter, M., et al,, [Effect of cytosine arabinoside on the differentiation of granulocyte- monocyte progenitors (CFU-GM) and myeloid leukemia blasts (CFU-L) In vitro]. Folia Haematol Int Mag Kiln Morphol Blufforsch, 1988. 115(6): p. 927-34.

46, Visani, G., et al., Effects of homoharringtonine alone and in combination with alpha interferon and cytosine arabinoside on ‘in vitro’ growth and induction of apoptosis in chronic myeloid leukemia and normal hematopoietic progenitors. Leukemia, 1997. 11(5): p. 624-8.

47. Ernst, T. J., L. N. Shulman, and M. Grossbard, Treatment of the chronic phase of CML with a combined continuous infusion of homoharringtonine and cytarabine. Blood, 1995. 86 suppl. 1 abstr. 2106.

48. Ernst, T. J., R. Soiffer, and R. Stone, Homoharringtonine with low dose cytarabine combination therapy induces both hematologic and cytogenetic remissions in patients with chronic myelogenous leukemia. Blood, 1996. 88 suppl. abstr. 2300.

49. Ernst, T. J., et al., Homoharringtonine and low-dose Ara-C is a highly effective combination for the treatment of CML in chronic phase. Blood, 1997. 90(10) suppl. 1 abs. 2305.

50. Kantarjian, H. M,, et al,, Homoharringtonine and low-dose cytarabine in the management of late chronic-phase chronic myelogenous leukemia. J Clin Oncol, 2000. 18(20): p. 3513-21.

51. Kantarjian, H. and e. al., Triple combination therapy with interferon-alpha(1IFN-A), low-dose cytarabine (LDara-C) and homoharringtonine (HHT) in Philadelphia chromosome (Ph)-positive chronic myelogenous leukemia (CML) in early chronic phase. Blood, 1999. Suppl. 1(Abstract 4440).

52. Feldman, E., et al., Homoharringtonine in combination with cytarabine for patients with acute myelogenous leukemia, Leukemia. 1992. 6(11): p. 1189-91.

53. Feldman, E. J., et al., Acute promyelocytic leukemia: a 5-year experience with new antileukemic agents and a new approach to preventing fatal hemorrhage. Acta Haematol, 1989. 82(3): p. 117-21.

54. Sacchi, S., et al., Chronic myelogenous leukemia in nonlymphoid blastic phase: analysis of the results of first salvage therapy with three different treatment approaches for 162 patients. Cancer, 1999. 86(12): p. 2632-41.

55. Xu, W. and M. Liang, [Chemosensitivity test for head and neck cancers]. Chung Hua Erh Pi Yen Hou Ko Tsa Chih, 1996. 31(4): p. 210-3.

1. A new method of therapy using the subcutaneous mode of administration of formulations based upon harringtonines including their salts and tautomeric forms having the formula 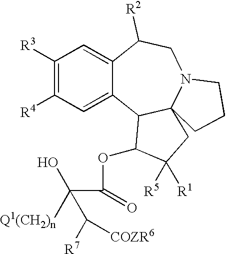
where:
R¹ is H, OH, OMe, O—(C₁-C₃₀)-alkyl, O-aryl-(C₁-C₃₀)-alkyl. O—(C₂-C₃₀)-alkenyl, O—(C₃-C₃₀)-cycloalkyl or null and
R² is H or OH, or R¹, R² form together —O—,
R³═R⁴═OMe or R³ and R⁴ form together —OCH₂O—,
n is 0 to 8
R⁵ is H, OH, OMe, O—(C₁-C₃₀)-alkyl, O-aryl-(C₁-C₃₀)-alkyl, O-(C₂-C₃₀)-alkenyl, O—(C₃-C₃₀)-cycloalkyl or O-aryl,
Z═O, S, or NH, and 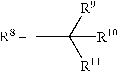
or Z—R⁸ is NR¹²R¹³, R¹² and R¹³ representing respectively R⁹ and R¹⁰,
R⁹, R¹⁰, R¹¹ are independently H, C₁-C₃₀ alkyl, C₃-C₃₀ cycloalkyl, aryl, aryl-(C₁-C₃₀)-alkyl, C₂-C₃₀ alkenyl, C₂-C₃₀ alkynyl, C₁-C₃₀trihalogenoalkyl, C₁-C₃₀alkylamino-(C₁-C₃₀)alkyl, C₁-C₃₀ dialkylamino(C₁-C₃₀)-alkyl, or amino-(C₁-C₃₀)-alkyl, or 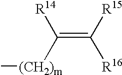
where R¹⁴, R¹⁵, R¹⁶ are independently H, halogen, C₁-C₃₀ alkyl, C₃-C₃₀ cycloalkyl, aryl, aryl-(C₁-C₃₀)-alkyl, C₂-C₃₀ alkenyl or C₂-C₃₀ alkynyl, C₁-C₃trihalogenoalkyl, m is 0to4,
each of these groups including or not heteroatom(s),
or their combination with another antitumor agent or a mixture of antitumor agents useful for the treatment of a disease in humans or animals, particularly cancers, leukemias, lymphomas, parasite diseases or chemotherapeutic resistance to other agents, in using a formulation specifically adapted for subcutaneous administration.

2. The method of claim 1 to 2 where the harringtonine is homoharringtonine or harringtonine having the following formula 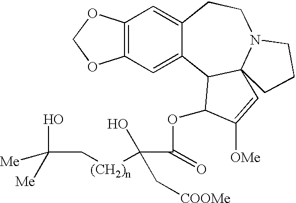
where n=1 or2

3. The method of claim 1 or 2 in which harringtonines are used under their sag forms having the following formula, 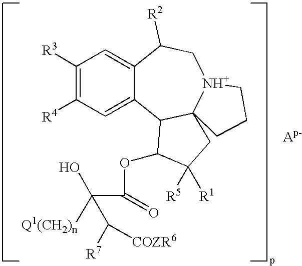
or, in particular the formula 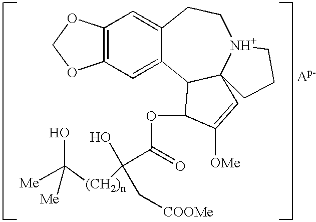
where A⁻ is a mineral anion such as chlorid, sulfate, nitrate, perchlorate or an organic ion such as tartarate, malate, lactate, or a citrate and p is 1 or 2.

4. The method of claims 1 to 3 in which the acid which forms a salt of harringtonines is hydrochlorid acid or tartaric acid.

5. The method of therapy of claims 1 to 4 in which the harringtonines are solution or hydrophilic freeze-dried powder ready-to-reconstitute of buffered salt of homoharringtonine or harringtonine of which the level of chromatographic purity suitable for medical use is higher than 99.7%

6. The method of therapy of claims 3 to 5 in which the pH of the formulation or constituted solution for injection is included between 5.5 and 8.

7. The method of therapy of claims 1 to 6 in which harringtonines are combined with another agent in the same injection.

8. The method of therapy of claim 7 in which the other agent is a nucleoside, preferably cytosine arabinoside

9. The method of therapy of claims 1 to 8 in which the subcutaneous mode of administration is performed by bolus injection at regular intervals such as one to four injection a day during 1 to n days for a cycle of n days, n being preferably 28.

10. The method of therapy of claims 1 to 8 in which the subcutaneous mode of administration is performed by continuous subcutaneous infusion.
